SEQUENCE LISTING (10 sequences)

SEQ ID NO: 1 - DNA, 42 bp, Artificial Sequence
  PCR primer
atcactaagctttatagacacacacgtgggcgcacacacaca

SEQ ID NO: 2 - DNA, 42 bp, Artificial Sequence
  PCR primer
tcacgaggtacccctgctcgtcggtcatcttcatgaaggggt

SEQ ID NO: 3 - DNA, 16 bp, Artificial Sequence
  PCR primer
gttgtggtttgtccaa

SEQ ID NO: 4 - DNA, 30 bp, Artificial Sequence
  PCR primer
actgcagaattcgccaagctcagatccaag

SEQ ID NO: 5 - DNA, 43 bp, Artificial Sequence
  PCR primer
atcactaagcttatgggagactgaagcaagaggatgcgataca

SEQ ID NO: 6 - DNA, 45 bp, Artificial Sequence
  PCR primer
tcactaggatccggcagccagggtgagcagaagaacgcaggcggt

SEQ ID NO: 7 - DNA, 33 bp, Artificial Sequence
  PCR primer
aagcttggatccgccttctataaaaggaaaagt

SEQ ID NO: 8 - DNA, 33 bp, Artificial Sequence
  PCR primer
ggtaccggcactggtactgcagctgggagggac

SEQ ID NO: 9 - DNA, 32 bp, Artificial Sequence
  PCR primer
cacacccaccagacaagcctgaacttgtctga

SEQ ID NO: 10 - DNA, 32 bp, Artificial Sequence
  PCR primer
aagccccagatgcgcagcctccagccaccgcc

(19) United States
(12) Patent Application Publication
(10) Pub. No.: US 2006/0003311 A1
(43) Pub. Date: Jan. 5, 2006

(54) In vitro screening of cellular events using 3d cell culture systems

(76) Inventors: Fabienne Andrea Fulde, Zurich (CH); Rupert Hagg, Bassasdorf (CH); Roberto Tommasini, Uster (CH)

Correspondence Address: LADAS & PARRY, 26 WEST 61ST STREET, NEW YORK, NY, 10023, US

(21) Appl. No.: 10/517,497

(22) PCT Filed: Jun. 12, 2003
(86) PCT No.: PCT/CH03/00377

§ 371 Date: May 26, 2005

(30) Foreign Application Priority Data
Jun. 13, 2002 (US) 60388483

(51) Int. Cl. C12Q 1/00; C12Q 1/68

(52) U.S. Cl. 435/4; 435/6

(57) ABSTRACT

The present invention relates to in vitro cell culture conditions wherein transfected cells containing promoter-reporter constructs are cultivated in a 3D tissue-like environment. The referred 3D culture conditions may eventually implement appropriate biomaterial or scaffolds.

### CROSS REFERENCES TO RELATED APPLICATIONS

This application claims the priority of provisional patent application 60/388,483, filed Jun. 13, 2002, the disclosure of which is incorporated herein by reference in its entirety.

### TECHNICAL FIELD

The present invention relates to in vitro cell culture conditions wherein transfected cells containing one or more selected promoter-reporter constructs are cultivated under conditions mimicking the natural in vivo environment. Such conditions may be achieved by providing 3D cell arrangements that optionally may include any scaffold or biomaterial.

The present invention further relates to a non-destructive and real-time assay for screening various cell types, preferably for cells of musculoskeletal tissues or cells being able to differentiate in such tissues, using key marker genes in form of novel promoter-reporter constructs that are transfected in said cells.

The present invention further presents small-scale in vitro cell culture conditions wherein said cell culture conditions have been adapted to various multiwell plates in order to enable higher throughput applications by an easy and convenient read-out with conventional standard plate readers or automated confocal microscope reader.

### BACKGROUND ART

General Information about Gene-Reporter Technologies

Cellular screening assays are widely used to study eukaryotic gene expression and cellular physiology such as ELISA-type assays, cellular Ca²⁺ assays, cellular assays based on novel fluorescence imaging methods or expression-reporter gene assays. Especially for reporter-gene technology the future is estimated to look bright (Naylor et al., 1999).

To a large extent, cellular screening techniques have been developed for pharmaceutical companies to cope with the steadily increasing amount of compounds. Various kinds of homogenous high throughput assay systems have enabled to increase the rate of sample processing (U.S. Pat. No. 5,989,835). Unfortunately, focus has mainly been laid on practicability rather than on biological relevance. As a consequence, almost all screening assays use in vitro culture conditions that do not mimic three-dimensional tissue-like arrangement as it occurs in vivo. For instance, disposable plastics have become the preferred substrate used in modern-day 2D cell cultures. While the growth of cells in two dimensions is a convenient method for preparing, observing and studying cells in culture, allowing a high rate of cell proliferation, it lacks the cell-cell and cell-matrix interactions characteristic of whole tissue in vivo. Therefore, these cells will start to loose their differentiated phenotype and become so called de-differentiated cells and at the same time will start to change their gene expression profile. As a consequence, mimicking tissue-like conditions by e.g. implementing an appropriate biomaterial or scaffold for cellular screening of a specific cell type is crucial for building up a functional relevant and meaningful assay. The invention described herein discloses such cell culture conditions that provide an excellent basis for functional cellular screening by combining appropriate natural or synthetic biomaterials together with a key set of novel promoter-reporter constructs.

Background Information Related to Cellular Screening with Cells from Musculoskeletal Tissues

Focus is being put more and more on cells that can be used to repair or regenerate musculoskeletal tissues such as bone and cartilage by means of tissue-engineering since they are structurally less complex than heart valves or entire organs such as pancreas and liver. In case of pathological conditions they may serve as cellular targets for screening of more effective drugs. Research efforts, either for tissue-engineering or for drug screening, in general have to consider differentiation pathways and subsequent maintenance of a differentiated phenotype. A series of factors (e.g. growth factors, pharmaceutical agents, biomaterials, mechanical stimulation and others) as well as their interactions are critical in this aspect. As a consequence, understanding of the exerted effects and the targeted control by using the appropriate factors in the appropriate concentration at the proper time point is far away from being realized. To overcome today's trial and error approaches, it is important to introduce novel and more sophisticated approaches to study this very complex subject matter.

For instance, two recent publications by Grant et al., 2000 and Bergwitz et al., 2001 have been proven to be valuable in tracking cellular responses of chondrocytes using the rat collagen type II (COL2) promoter in combination with green fluorescent protein (GFP). While Grant et al. generated a COL2-GFP reporter mouse model as a new tool to study skeletal development by marking the chondrocyte lineage and chondrogenesis; Bergwitz et al. established a stable transfected chondrocytic cell line and sandwich co-cultures where wnt-secreting cells in monolayer were overlaid by agarose suspension cultures containing the rat COL2-GFP transfected cell line. In this manner, it was possible to examine the effect of wnt-proteins on the early chondrocyte differentiation. Beyond dispute of the scientific impact of these publications, broad and simple application of these models is not possible for several reasons. Only a few laboratories have the tools to produce transgenic mice, to keep them in animal houses, or analyze them appropriately. Common use is further restricted through a limited number of experiments, a limited number of promoter-reporter genes and the restriction to mouse species. The chondrogenic rat calvaria cell line eliminates the above-mentioned restriction with respect to the number of experimental testing. However, the drawback of only one promoter-reporter gene construct and the restriction to a rodent derived cell line still remains. The narrow scope of application is further underlined by a complex 3D co-culture model requiring quite a high cell number which is not suitable for fast and convenient read-outs.

While U.S. Pat. No. 5,932,459 and other literature references, e.g. Stokes et al., 2001, Hauselmann et al., 1994, in principle describe culture systems that could be used to study the re-differentiation process of cells they do not allow to be applied within high throughput screening applications since they all require out of scale quantities of a characterized cell source. Also the possibility of a read-out process by using conventional plate-readers for such cell-based assays has not been addressed.

Furthermore, since all ex vivo tissue-engineering approaches include a cell source as one major, if not the most important element, appropriate quality control of current commercialized products is becoming more and more a central issue. Beyond viability and sterility, it is desired to receive information about the cellular status of the cells that are used directly for a cellular therapy or being used to form de novo tissue following cultivation in 3D.

All these drawbacks have precipitated the concept of monitoring gene expression of key marker genes within various more functionally relevant culture conditions that allow fast and simple read-out, preferably in a non-destructive and real-time manner.

The current invention offers a new tool to study marker (positive and negative) gene expression and thus to determine whether the desired cellular phenotype is maintained.

### DISCLOSURE OF THE INVENTION

In a first aspect, the present invention provides a screening method for compounds having a modulating effect on cellular development and/or cell differentiation and/or cellular processes. Said screening method comprises the following steps:
a) cultivating cells harboring a promoter-reporter construct in a 3D micro-culture under conditions mimicking the natural environment (3D tissue-like) of said cells, or cultivating said cell in a 2D culture on bioinductive material,
b) contacting said cells with a test compound and comparing the read-out of the promoter-reporter construct to a control.

In a preferred embodiment of the present invention said 3D culture comprises a biomaterial substrate or scaffold that promotes normal physiological activity, in particular scaffolds/biomaterials selected from the group of natural scaffolds/biomaterials consisting of alginate, agarose, hyaluronic acid, collagen, proteoglycan, mixtures thereof or from the group of synthetic scaffolds/biomaterials consisting of Skelite™, polyHEMA, polyglycolic acid (PGA), polylactic acid (PLA), mixtures of PGA and PLA.

In a further preferred embodiment said cells are selected from the group consisting of chondrocytes, bone cells, rheumatoid cells, osteoarthritic chondrocytes, stem cells, mesenchymal cells, cartilage or bone tumor cells, preferably said cells stem from humans.

In yet a further preferred embodiment said promoter is selected from the group consisting of COL1, COL2, SOX9, COMP, MMP2 and aggrecanase-1.

In a further preferred embodiments said reporter is selected from the group of GFP, luciferase, β-galactosidase, chloramphenicol acetyltransferase gene (CAT).

Preferred cells for use in a method of the present invention stem from humans and said promoter-reporter construct is a DNA construct of the present invention.

In a preferred embodiment, said cells comprise more than one promoter-reporter construct.

Test compounds are preferably selected from the group consisting of chemical libraries, natural product libraries, peptide libraries, cDNA libraries and combinatorial libraries.

In a much preferred embodiment of the present invention the method is performed in a multiplate culture format e.g. 96 or 384-mulitwells.

In a further preferred embodiment said cells are contacted with an activator or suppressor of said promoter and with a test compound.

In a second aspect, the present invention relates to a DNA construct for cell transfection. Said DNA construct comprises a reporter gene under control of a human promoter wherein said promoter is selected from the group consisting of human COL1, human COL2, human SOX9, human COMP, human MMP2 and human aggrecanase-1 and said reporter gene encodes a protein with an activity that can be detected by colorimetric or fluorescent methods.

In a preferred embodiment said reporter is selected from the group consisting of GFP, luciferase, β-galactosidase, chloramphenicol acetyltransferase gene (CAT).

In a further aspect, the present invention relates to a method for testing whether a material has bioinductive characteristics. Said method comprises the following steps:
culturing cells harboring a promoter-reporter construct on the material to be tested and
comparing the read-out of the promoter-reporter construct to a control.

In a further aspect, the present invention relates to a method for testing whether a biomaterial is degraded or resorbed in vivo or in vitro. Said method comprising the following steps:
culturing cells harboring a promoter-reporter construct on the material to be tested and
monitoring expression of the reporter gene in said cells.

In a further aspect, the present invention relates to a use of a promoter-reporter construct for the construction of transgenic animals, preferably transgenic mice. Said construct comprises a reporter which is selected from the group consisting of GFP, luciferase, β-galactosidase, chloramphenicol acetyltransferase gene (CAT) and a promoter which is selected from the group consisting of COL1, COL2, SOX9, COMP, MMP2 and aggrecanase-1.

The resulting transgenic animals can be used in a screening method for compounds having a modulating effect on cellular development and/or cell differentiation and/or cellular processes.

In a further aspect the present invention relates to cells or cell lines comprising a reporter construct of the present invention. Said cells or cell lines are preferably selected from the group consisting of chondrocytes, bone cells, rheumatoid cells, osteoarthritic chondrocytes, stem cells, mesenchymal cells, cartilage or bone tumor cells, preferably said cells stem from humans.

In a further aspect, the present invention provides a method for the quality control of cells cultivated in vitro. Said method comprises the following steps:
transfecting cells that have been cultured in vitro with a key marker promoter-reporter construct and cultivating said transfected cells in a 3D culture and
detection of the reporter read-out which is indicative for differentiated cells.

Cells used in said methods are preferably of human origin, preferably cells that belong to the groups as defined herein before. A preferred reporter and preferred promoter for use in said method is selected from the groups defined herein before.

The present invention provides a novel cell-based screening assay and variants thereof that may optionally include biomaterials and scaffolds, either natural or synthetic ones to mimic the environmental nature of a given tissue within cell culture conditions on a small-scale level. Most preferably, the disclosed cell culture conditions in combination with transfected cells is suitable to cultivate cells derived from musculoskeletal tissues or cells being able to differentiate in such tissues.

The present invention also encompasses various novel human transcriptional promoter-reporter constructs, preferably with those promoters that are regarded as key markers for a specific cell type, e.g. collagen type II, SOX9 and COMP for chondrocytes, or a specific cellular status, e.g. aggrecanase-1 (ADAMTS4) and MMP2 for osteoarthritic cells. These gene-reporter constructs, preferably with the most common luminophore reporters such as GFP or luciferase allowing non-invasive monitoring of gene expression, can be used in combination with the disclosed 3D in vitro cell culture conditions to evaluate the influence of signaling molecules, drugs or other medium components on proliferation, differentiation or de novo tissue formation. By using pluripotent stem cells or progenitor cells, monitoring along a differentiation pathway or commitment to a specific lineage is possible, most preferably for those cells differentiating into musculoskeletal tissues. Novel functional data about genes having a regulatory role within any of the mentioned cell types can be achieved by e.g. co-transfection of any cell type with gene libraries containing CMV driven cDNA sequences.

A further important aspect of the invention is the specific adjustment of the described 3D culture conditions to conventional microtiter plates, e.g. 96 or 384-well format allowing reliable and accurate read-outs with the most important reporter genes such as GFP and luciferase.

A further important aspect of the invention discloses the possibility of the described invention to be used as a new quality control tool and/or diagnostic tool to enhance clinical outcome of cellular/tissue-engineered therapies.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1, time dependent curve for 2×10⁵ adenovirus infected porcine chondrocytes (P2) expressing CMV-GFP in 3D alginate disc culture within a 96-well plate over time, transfection rate 90%. GFP expression measured with BMG Fluostar, 485/20 nm excitation filter and 535/20 nm emission filter during 12 days. ⋄ transfected cells, ▪ control cells.

FIG. 2, cell number dependent curve for adenovirus infected porcine chondrocytes (P2) expressing CMV-GFP in 3D alginate disc culture within a 96-well plate, transfection rate 90%. GFP expression measured with BMG Fluostar, 485/20 nm excitation filter and 535/20 nm emission filter at day 2.

FIG. 3, adenovirus infected porcine chondrocytes (P2) expressing CMV-GFP in 3D alginate disc culture within a 96-well plate. a) Image taken by light microscope on day 3. b) Image taken by fluorescence microscope with a 470/40 nm excitation filter, a 495 nm beamsplitter and a 525/50 nm emission filter on day 3.

FIG. 4, calcein AM/propidium iodide life/dead staining of untransfected porcine chondrocytes (P2) in 3D alginate disc culture within a 96-well plate. a) Image taken of living cells by fluorescence microscope with a 470/40 nm excitation filter, a 495 nm beamsplitter and a 525/50 nm emission filter on day 12. b) Image taken of dead cells by fluorescence microscope with a 535/50 nm excitation filter, a 580 nm beamsplitter and a 590LP nm emission filter on day 12.

FIG. 5, time dependent curve for 2×10⁵ transfected porcine chondrocytes (P2) expressing CMV-GFP in 3D agarose disc culture within 96-well plate over time, transfection rate 90%. GFP expression measured with BMG Fluostar, 485/20 nm excitation filter and 535/20 emission filter during 13 days. ⋄ transfected cells, ▪ control cells.

FIG. 6, cell number dependent curve for adenovirus infected porcine chondrocytes (P2) expressing CMV-GFP in 3D agarose disc culture within a 96-well plate, transfection rate 90%. GFP expression measured with BMG Fluostar, 485/20 nm excitation filter and 535/20 nm emission filter at day 2.

FIG. 7, adenovirus transfected porcine chondrocytes (P2) expressing CMV-GFP in 3D agarose disc culture within a 96-well plate. a) Image taken by light microscope on day 1. b) Image taken by fluorescence microscope with a 470/40 nm excitation filter, a 495 nm beamsplitter and a 525/50 nm emission filter on day 1.

FIG. 8, calcein AM/propidium iodide life/dead staining of untransfected porcine chondrocytes (P2) in 3D agarose disc culture within a 96-well plate. a) Image taken of living cells by fluorescence microscope with a 470/40 nm excitation filter, a 495 nm beamsplitter and a 525/50 nm emission filter on day 13. b) Image taken of dead cells by fluorescence microscope with a 535/50 nm excitation filter, a 580 nm beamsplitter and a 590LP nm emission filter on day 13.

FIG. 9, time dependent curve for 2×10⁵ with Amaxa Nucleofector™ technology transfected human chondrocytes (P2) expressing CMV-GFP in 3D agarose disc culture within a 96-well plate over time, transfection rate 40%. GFP expression measured with BMG Fluostar, 485/20 nm excitation filter and 535/20 nm emission filter during 10 days. ⋄ transfected cells, ▪ control cells.

FIG. 10, cell number dependent curve for Amaxa Nucleofector™ technology transfected human chondrocytes (P2) expressing CMV-GFP in 3D agarose disc culture within a 96-well plate, transfection rate 40%. GFP expression measured with BMG Fluostar, 485/20 nm excitation filter and 535/20 nm emission filter at day 2.

FIG. 11, cell number dependent curve for Fugene6 transfected porcine chondrocytes (P2) expressing COL1-luciferase in 3D agarose disc culture within a 96-well plate, transfection rate 15%. Luciferase expression measured with a Berthold Detection System MPL2 luminometer at day 1.

FIG. 12, time dependent curve for 2×10⁵ adenovirus infected porcine chondrocytes (P2) expressing CMV-GFP seeded on polyHEMA within a 96-well-plate, transfection rate 90%. GFP expression measured with BMG Fluostar, 485/20 nm excitation filter and 535120 nm emission filter during 10 days. ⋄ transfected cells, ▪ control cells.

FIG. 13, cell number dependent curve for adenovirus infected porcine chondrocytes (P2) expressing CMV-GFP seeded on polyHEMA within a 96-well plate, transfection rate 90%. GFP expression measured with BMG Fluostar, 485/20 nm excitation filter and 535/20 nm emission filter at day 3.

FIG. 14, adenovirus infected porcine chondrocytes (P2) expressing CMV-GFP seeded on polyHEMA in a 96-well plate. a) Image taken by light microscope on day 1. b) Image taken by fluorescence microscope with a 470/40 nm excitation filter, a 495 nm beamsplitter and a 525/50 nm emission filter on day 5.

FIG. 15, adenovirus infected osteoarthritic human chondrocytes (P2) expressing CMV-GFP, 16 hours after infection. Image taken by fluorescence microscope with a 470/40 nm excitation filter, a 495 nm beamsplitter and a 525/50 nm emission filter.

FIG. 16, growth curve of primary human articular chondrocytes (P2) cultured on Osteologic™ discs and standard tissue culture plastic. Cell counts by trypan blue exclusion method at day 0, 2, 4 and day 7. ⋄ cells on Osteologic™ disc, ▪ cells on standard tissue culture plastic.

FIG. 17, growth of human chondrocytes on standard tissue culture plastic vs. Osteologic™ discs. Passage 3 cells stained with PAS stain at day 7. a) human chondrocytes on standard tissue culture plastic. b) same cells grown on Osteologic™ disc.

FIG. 18, Amaxa Nucleofector™ technology transfected human chondrocytes (P0) expressing SOX9-GFP, transfection rate 35%, showing functionality of the cloned promoter-reporter construct. Image taken by fluorescence microscope with a 470/40 nm excitation filter, a 495 nm beamsplitter and a 525/50 nm emission filter.

FIG. 19, Amaxa Nucleofector™ technology transfected human chondrocytes (P0) expressing COL1-GFP, transfection rate 18%, showing functionality of the cloned promoter-reporter construct. Image taken by fluorescence microscope with a 470/40 nm excitation filter, a 495 nm beamsplitter and a 525/50 nm emission filter.

FIG. 20, Amaxa Nucleofector™ technology transfected human chondrocytes (P0) expressing COL2-GFP, transfection rate 10% showing functionality of the cloned promoter-reporter construct. Image taken by fluorescence microscope with a 470/40 nm excitation filter, a 495 nm beamsplitter and a 525/50 nm emission filter.

FIG. 21, Amaxa Nucleofector™ technology transfected human chondrocytes (P0) expressing COMP-GFP, transfection rate 39%, showing functionality of the cloned promoter-reporter construct. Image taken by fluorescence microscope with a 470/40 nm excitation filter, a 495 nm beamsplitter and a 525/50 nm emission filter.

FIG. 22, Amaxa Nucleofector™ technology transfected human chondrocytes (P0) expressing aggrecanase-1 (ADAMTS4)-GFP, transfection rate 37%, showing functionality of the cloned promoter-reporter construct. Image taken by fluorescence microscope with a 470/40 nm excitation filter, a 495 nm beamsplitter and a 525/50 nm emission filter.

FIG. 23, Amaxa Nucleofector™ technology transfected human chondrocytes (P0) expressing MMP2 short-GFP, transfection rate 15%, showing functionality of the cloned promoter-reporter construct. Image taken by fluorescence microscope with a 470/40 nm excitation filter, a 495 nm beamsplitter and a 525/50 nm emission filter.

FIG. 24, Amaxa Nucleofector™ technology transfected human chondrocytes (P0) expressing MMP2 long-GFP, transfection rate 17%, showing functionality of the cloned promoter-reporter construct. Image taken by fluorescence microscope with a 470/40 nm excitation filter, a 495 nm beamsplitter and a 525/50 nm emission filter.

### MODES FOR CARRYING OUT THE INVENTION

Definitions

Bioinductive: refers to a natural or synthetic biomaterial that influences cells in such a way to preserve or induce a differentiated phenotype, even in the absence of exogenously added growth factors. Skelite™ is a good example for a bioinductive or osteoinductive material.

Non-destructive/non-invasive: refers to an assay and allows the measurement of key parameters without destroying the current cell culture.

Real-time: refers to a direct measurement of signals produced by reporter molecules related to the cell-based assay described in the current invention

3D: refers to a cell culture system where cells are kept in a three-dimensional condition to provide a tissue-like environment and therefore allows to preserve or induce a differentiated phenotype of the cells.

3D micro-cultures: refers to three dimensional cell culture conditions optionally including biomaterials or scaffolds where cells are kept in a tissue-like environment which preserves or induces a differentiated phenotype of the cultivated cells and requires only a limited amount of cells in order to qualify for high throughput applications.

2D: refers to the expansion of cell cultures in an anchorage dependent condition on the surface of a plastic or any other biomaterial substrate.

Promoter-reporter constructs: are various constructs where a promoter or a transcriptional element thereof is linked to reporter molecules such as green fluorescent protein or luciferase to perform real-time and non-destructive measurements in cell cultures.

Skelite™ (Millenium Biologix Inc., Canada): is a synthetic bioactive bone biomaterial on basis of calcium phosphate. This exceptional biological performance is based on a chemical composition and physical structure that mimics natural bone.

Read-out: in the present context, the term read-out is used for qualitative and quantitative assessment of signals produced by reporter molecules that are e.g. detected by a conventional standard fluorescence plate reader or a fluorescence microscope. Since cell culture parameters have been adapted to various multiwell plates, easy and convenient read-out through conventional standard plate readers has been achieved. This allows statistical determination of parameters such as accuracy, reproducibility and detection limit. These are important aspects for the adaptation to high throughput systems in drug screening applications.

GFP: in the present context, the term “green fluorescent protein” is intended to indicate a protein which, when expressed by a cell, emits fluorescence upon exposure to light of the correct excitation wavelength (Chalfie et al. 1994).

Luminophore: the luminophore is the component that allows to be visualized and/or recorded by emitting light related to the degree of influence.

### DETAILED DESCRIPTION OF THE INVENTION

The present invention provides a novel cell-based screening assay on basis of gene-reporter technology and variants thereof that may optionally include biomaterials and scaffolds, either natural or synthetic ones, to mimic the environmental nature of a given tissue within cell culture conditions on a small-scale level. Thus, more functional screening of cellular events will be feasible on a high throughput level. Most preferably, the disclosed cell culture conditions in combination with transfected cells are suitable to cultivate cells derived from musculoskeletal tissues or cells being able to differentiate in such tissues.

While growth and maintenance of cells in 3D culture provide cell-cell and cell-matrix interactions as they occur in vivo, growth of cells in 2D culture lacks these interactions and represents therefore a rather artificial situation. Hence, such culture conditions are only suitable for proliferation studies and not for experimental conclusions requiring a differentiated phenotype. Although this is well known, most commercial cellular assays including gene-reporter assays are based on cultivation of cells on plastic culture flasks. In limited cases it may become possible to improve the experimental situation of 2D cultures by simply replacing the artificial plastic culture substrate by a thin layer of a more appropriate biomaterial. This may then allow to circumvent the need of much more complex 3D cell culture conditions. A good example for this is the Osteologic™ (Millenium Biologix Inc., Canada) bone cell culture system which consists of sub-micron synthetic calcium phosphate thin films coated onto various culture vessels. Thus, it has been demonstrated to assess osteoclast/osteoblast activity and growth in a more biological relevant manner. Recent studies have shown that beyond bone cells also chondrocytes grow significantly better on Osteologic™ substrate than on traditional tissue culture plastic.

Another important aspect of the present invention are various 3D culture conditions allowing to screen cellular behavior of cells, most preferably those of musculoskeletal tissue or precursor cells under conditions that strongly support differentiation along a desired lineage pathway or maintain the corresponding fully differentiated phenotype over an extended time in culture. By transfecting the mentioned cell types with a corresponding gene-reporter construct such as described in this invention, monitoring of cell differentiation and commitment to the respective cell lineage is possible. Such screening assays will also aid in the development of drug candidates or drug targets by elucidating the function of those drugs or genes during differentiation along the lineage pathway. Alternatively, by using already differentiated cells of healthy tissue and comparing those with cells from pathological tissue such as cells derived from tissues of arthritic joints, highly efficient screening of drugs can be achieved. The 3D culture system of the embodiment may include some natural or synthetic scaffold material like alginate, agarose, hyaluronic acid, Skelite™ or any other material providing a three dimensional structure where cells can communicate with each other via autocrine and paracrine factors as well as have the appropriate feedback from extracellular substances such as they occur in vivo.

The invention also encompasses the downscale or adjustment of the described 3D culture conditions to multiplate culture formats, e.g. 96 or 384-multiwells, such as these 3D culture conditions for cellular screening may contain only a few cells up to a couple of thousands per culture system and qualify for high throughput applications by providing a readily machine readable signaling with e.g. standard plate readers. This is of major advantage since 3D cultures often require a large amount of cells and are therefore per se not applicable for efficient screening of agents, if biological more relevant primary cell sources shall be the basis for cellular screening.

The current invention will therefore provide a platform for small-scale 3D tissue cell culture systems by combining proper biomaterials together with key marker promoter-reporter constructs. Through this downscale costly culture material, cells and testing substances, e.g. growth factors, hormones or any other culture media components, can be saved. Another major benefit is the possibility to adjust such small-scale on-line and nondestructive screening assays to commonly used multiwell plates (96 to 384-well plates) to achieve fast, simple and convenient “reporter read-out” by means of conventional standard plate readers besides other analytical tools, e.g. a fluorescence microscope.

The current invention also provides a new set of human promoter-reporter constructs that can be used to transfect primary cells, cell lines or even to prepare transgenic animals such as transgenic mouse lines that express the reporter under the control of a promoter. The scope of this part of the invention is described in the following by means of a few examples.

The current invention provides a method for screening agents as candidates for drugs or growth factors for enhancing the formation of new cartilage tissue in vitro. Cells transfected with a construct comprising the human COL2 or equivalent thereof, e.g. synthetic equivalent thereof, or in combination with the human COLL promoter or any other promoter element ligated to distinct reporter gene, are cultivated in 3D systems and treated with an activator of the COL2 promoter. The agent being screened may be tested for its ability to stimulate the COL2 promoter. The agent is a candidate as a drug or source of a drug being able to induce collagen type II expression in vitro and to increase new tissue formation. While collagen type II is being stimulated by the screened drug, COL1 expression in contrast will be reduced since it is a negative marker for the differentiated phenotype of hyaline cartilage. While a recent paper published by Bergwitz et al., 2001 describes the transfection of chondrocytes with a rat promoter of collagen type II, one has to realize that promoter elements may be very species specific. While some elements of the COL2 promoter will be necessary in the rat some of the same regulatory elements will not be needed in human or vice versa. Therefore, even if highly identical sequential regions exist on both promoters they may behave in a complete different way in vivo. This is fundamental and may have strong effects when agents are screened in cultures and may prevent the discovery of new lead compounds or negatively influence lead optimization that may not be obvious. This invention therefore preferably applies to the use of human promoter elements transfected in the background of human cells.

In accordance with a further embodiment the invention provides a method for the assessment of the chondrocyte phenotype by using promoter elements of the human SOX9 and COMP genes. Both genes are chondrogenic markers that can be used to indicate chondrogenic differentiation of precursor cells or to detect the recovery and maintenance of the differentiated phenotype of articular chondrocytes follwowing expansion in 2D culture. Cell cultures transfected with the above promoter-reporter constructs will be screened with agents that may induce and/or maintain the differentiated phenotype in vitro.

The current invention discloses also a method for screening agents as candidates for drugs for prophylaxis or treatment of mammalian disorders caused or mediated by aggrecanase-1 (ADAMTS4) expression. Thus, cells may be transfected in a cell background that strongly induces aggrecanase-1 expression e.g. rheumatoid or osteoarthritic cell sources transfected with a construct containing a transcriptional promoter element from the human aggrecanase-1 gene or equivalent thereof, e.g. synthetic equivalent thereof, ligated to a reporter gene and cultivated as 3D micro cultures optionally on/within a biomaterial/scaffold. Another experimental set-up would include healthy chondrocytic cells transfected with a construct containing a transcriptional promoter element from the human aggrecanase-1 gene or equivalent thereof, e.g. synthetic equivalent thereof, ligated to a reporter gene and cultivated as 3D microstructures optionally on/within a biomaterial/scaffold and treated with an inducer of the aggrecanase-1 promoter activity e.g. interleukin 1. The agent being screened is then tested for its ability to suppress promoter activity. The agent is a candidate as a drug or source of a drug for prophylaxis or treatment of mammalian disorders caused or mediated by aggrecanase-1 expression if the agent reduces stimulated promoter activity. Where an agent is determined to inhibit stimulation of aggrecanase-1 promoter, this indicates a higher likelihood of inhibiting any degradation of cartilage matrix.

The current invention discloses also a method for screening agents as drug candidates for prophylaxis or treatment of mammalian disorders caused or mediated by expression of matrix metalloproteinases (MMPs). MMP's e.g. MMP2 play an important role in the evolution of joint erosions in patients with non-inflammatory osteoarthritis and inflammatory rheumatoid arthritis. The gelatinase MMP2 has further shown to be involved in cancer, above all in tissue-invasive metastatic diseases.

MMP promoter elements linked to reporter molecules like e.g. GFP can thus be used not only to study cartilage degenerative processes taking place in arthritic conditions but also can be applied to study the obstacles of cancer development and progression via metastasis formation. In both processes the degradation of the extracellular matrix is taking place and this process can be best studied by using biological relevant cell culture conditions where the cells behave similar to the in vivo situation. Cells may then be transfected into a cell background that strongly induces MMP expression e.g. rheumatoid or osteoarthritic. or tumor cell sources, with a construct containing a transcriptional promoter element from the human MMP2 gene or equivalent thereof, e.g. synthetic equivalent thereof, ligated to a reporter gene and cultivated as a 3D micro culture optionally on/with a biomaterial/scaffold. This cell culture system may then be screened with agents that may suppress the expression of the reporter molecule, indicative for a molecule that will allow to reduce MMP expression also in vivo. Another experimental set-up would include e.g. primary human cells isolated from healthy cartilage tissue and transfected with a construct containing a transcriptional promoter element from the human MMP gene or equivalent thereof, e.g. synthetic equivalent thereof, ligated to a reporter gene and grown optionally on/within a biomaterial/scaffold and treated respectively with an inducer of MMP promoter activity e.g. Tumor Necrosis Factor α. The agent being screened is then tested for its ability to suppress stimulation of the promoter and a potential candidate as a drug or source of a drug for prophylaxis or treatment of mammalian disorders from cartilage degeneration.

While the reporter molecule will preferentially be firefly luciferase and GFP or any other fluorescence molecule, other reporter systems for use for this purpose include, for example beta-galactosidase gene (beta.gal) and chloramphenicol and acetyltransferase gene (CAT). Assays for expression produced in conjunction with each of these reporter gene elements are well-known to those skilled in the art. By preferentially having luciferase and GFP as reporter molecule, the advantage is of being able to perform real-time follow-up studies on cell cultures without the need to destroy the cells. A further advantage by having reporter molecules that allow nondestructive measurement is to be able to perform temporal and spatial analysis, a topic that is of major relevance when tissue-engineered constructs are grown in vitro. This allows monitoring cell relevant marker gene expression in these cell culture systems in a real-time and non-destructive manner and to determine whether the cells in the grown tissue are equally differentiated and well nourished. Especially when having 3D cultures that are cultivated over an extended time, e.g. four weeks as it is the case in the field of cartilage tissue-engineering, it may be of great advantage to follow the development of the same tissue in vitro without destroying the material.

The current invention does not only cover the aspect of having single promoter-reporter elements in one cell. The combination of several vectors containing one or more promoter elements in the same cells e.g. cotransfection with cDNA libraries in the same cell may also be disclosed. This may be of major importance when screening new proteins that may act as inductors or repressors on the reporter construct to be tested. In such a screening process libraries containing expression vectors where cDNA are linked to a constitutive promoter like e.g. CMV may be co-transfected with to be analyzed promoter-reporter construct and screened for the induction or repression of the reporter molecules. This will allow to detect new target molecules e.g. transcription factors and to identify new lead compounds for clinical applications.

The current invention also encompasses cell lines that are derived from the above mentioned transfection or co-transfection experiments, these cell lines can then be used as standard elements during further screening processes for the discovery of new molecules.

The current invention has disclosed a novel cell-based screening tool that may be applicable for screening of drugs, growth factors or any other beneficial components during development of cellular or tissue-engineered therapies. It does not matter whether the donor cells are from an autologous, allogeneic, xenogeneic cell source or whether the cells are non-differentiated precursor cells or already fully differentiated cells. Furthermore, an in vitro screening system that allows to be performed on miniaturized 3D tissue-like cultures has not been disclosed before and enables a more reliable validation of cellular targets, to assess more precisely toxicological responses and to increase the probability of success of new leads in the clinic.

While U.S. Pat. No. 6,200,760, U.S. Pat. No. 6,083,690 and U.S. Pat. No. 6,338,944 describe methods of screening agents in combination with gene promoter-reporter constructs, these inventions have not properly addressed the biological relevance of any cellular screening event, above all in context with the important issue to simultaneously allow accurate and reliable read-out on a higher throughput level. Other patents like U.S. Pat. No. 5,858,721 have disclosed the method of 3D system cultivation for tissue-engineering applications but have not considered the application of using such systems within micro scale cultures to be used within 96 or 384-well

Therefore, applications may even include the possibility to screen the toxicity of new chemicals and drugs as an important alternative to animal models for e.g. the cosmetic industry. By cultivating cell populations in a three dimensional system new drugs or molecules can be tested more thoroughly since a tissue-like system is provided. Cell-based screening tools may be the preferred technique in drug discovery, because it generates leads with a higher probability of progressing to clinical trials. Another important aspect includes the determination of dose response curves for new drugs and can be useful in the field of pharmakokinetics. Cells isolated from a patient and cultured under 3D conditions disclosed in the invention may then be used to assess further treatment by choosing the best of a selection of drug molecules. Furthermore, cells can be isolated on later stage and checked for disease progression. Therefore, the current invention relates to the application for cell-based diagnostics.

Another important aspect of the current invention is the use of the screening assay as a quality control for cell/tissue-based therapies for product and material testing. Because compendial methods do not yet exist, meaningful assays are required and need to be validated to monitor performance of key components such as the cell source or any biomaterial to be included. The herein described assay may be especially suitable for determining the cell potency of any cell source, e.g. autologous, allogeneic, xenogeneic or genetically engineered cells. A critical test could be to ascertain the necessary proliferative and/or differentiation capability of the cell source. Within any cell therapeutic approach such as autologous chondrocyte implantation (ACI) it would be possible to monitor the differentiation ability after lot release of the product and to better control or assess the clinical outcome of such a therapy. In case of a tissue-engineered product that requires further cultivation in 3D for a certain time period following cellular expansion, a screening assay of this invention could be used, e.g. along with other quality control tests as a checkpoint for lot release of the final implant.

Yet another important aspect of the described invention is the use of this screening assay as a diagnostic tool. In this sense, donor cells from autologous, allogeneic, or xenogeneic sources, e.g. healthy living adults, fetals, and/or cadavers may be checked for their suitability (cell potency) within a cell/tissue-engineered therapy. Further, the corresponding cells may be analyzed in the clinic for their proliferative and/or differentiation ability in order to decide on the most promising therapy. This may be a cellular therapy, a tissue-engineered therapy, or in case of a negative diagnosis with the disclosed assay a traditional surgical approach. It may also be the case that the diagnostic assay will monitor the cellular status of the donor source and in case of any pre-determined deficiencies correct these by e.g. adding the required growth factor(s), hormones, pharmaceutical agent etc.

A further application of the current invention may include the assessment of the performance of biomaterials in combination with cells or tissue. Cells or tissues containing transfected cells with appropriate promoter-reporter constructs may be used to assess the inductive potential of biomaterials regarding their potential of inducing new tissue formation or preserving the differentiated phenotype. Biomaterials that will positively influence the cultivated cells with respect to inducing differentiation or preserving the correct phenotype may show a higher expression of the reporter molecule according to the selected marker promoter. This may then be indicative of a positive feedback of the material to the cell and will help to better design and adapt new materials to the corresponding cells or tissue.

In yet another aspect, the biomaterials coated with a key marker promoter-reporter construct may be used to assess the degradation or resorption of the biomaterial in vivo or in vitro. When biomaterials are resorbed in vivo or in vitro plasmid released from the material will transfect surrounding cells. If an adequate promoter-reporter molecule is used the surrounding cells will then express a reporter molecule e.g. GFP indicative for the released vector molecules and resorption and degradation can be studied.

The current invention may also be used to study new in vitro tissue formation on a larger scale by using transfected cells with selected promoter-reporter molecules. These cells may then be grown in vitro or in vivo and tissue formation can be assessed by determining the expression of the reporter molecule. A similar experimental setup may be used and performed in animal model, were transfected cells with corresponding promoter-reporter constructs may be included in the transplanted tissue to follow the development of the tissue in vivo.

The invention is now further described by means of examples.

### EXAMPLE 1

3D Micro Cell Culture Models Mimicking a Cartilage Tissue-Like Environment

Useful for 3D culture conditions that can be downscaled to e.g. 96 or 384-well format, suitable for e.g. high throughput screening applications or to be applied as a quality control tool within cell-based therapies.

Cell Isolation and Propagation

Articular cartilage was harvested from healthy young (6 months) pigs or human donors (age 56 and 79 years). Minced cartilage pieces were digested with 0.025% (weight/volume) collagenase and 0.015% (weight/volume) pronase in DMEM/F-12 containing 5% fetal calf serum (FCS), 73 μg/ml ascorbic acid, 100 IU/100 μg/ml penicillin/streptomycin, 1 μg/ml insulin, 50 μg/ml gentamycin, 1.5 μg/ml amphotericin B, 2.5% Hepes buffer for 16 hours at 37° C. in 5% CO₂. Isolated chondrocytes were spun, resuspended in complete medium, counted and plated at a density of 5×10⁶ cells per cm². Cells were routinely passaged at confluence (every 5-7 days). Proliferation medium was DMEM/F-12 containing 10% FCS, 14.5 μg/ml ascorbic acid and 50 IU/50 μg/ml penicillin/streptomycin.

Transfection and 3D Cell Culture Conditions

To prepare transfected cells that will be used for micro 3D cultures, three different transfection methods were applied.

a) Viral Infection Using Adenovirus (AV)

5×10⁴ chondrocytes per 24-well were infected with AV (MOI=100) containing GFP under the control of a CMV promoter for 16 hours in DMEM/F-12 containing 2% FCS, 14.5 μg/ml ascorbic acid and 50 IU/50 μg/ml penicillin/streptomycin.

b) Amaxa Nucleofector™ technology

Human chondrocytes were transfected with pGFP-CMV using Amaxa Nucleofector™ technology. Briefly, 5 μg plasmid were mixed with 5×10⁵ cells in 100 μl nucleofection solution and subsequently nucleofected using program U-24 from Amaxa Nucleofector™ technology. Transfected cells were plated in a 6-well plate, medium was changed after 24 hours.
c) Lipid Based Transfection Method Using Fugene6

2.5×10⁵ cells were transfected in a 6-well plate using Fugene6, Roche, Switzerland, with a plasmid containing the luciferase gene under the control of a collagen type I promoter, kindly provided by F. Ramirez, New York. 3 μl reaction reagent per 1 μg DNA was used. Transfection reagent was removed after 24 hours. To determine transfection rate, a co-transfection with pGFP-CMV was performed.

Subsequently, cells were detached from monolayer culture and put in one of the following tissue-like culture cultivation methods described below. In all cases, cells were then maintained in differentiation medium DMEM/F-12 with 10% FCS, 1 μg/ml Insulin, 73 μg/ml ascorbic acid at 37° C. and 5% CO₂. Untransfected chondrocytes were used as control.

Monitoring GFP Expression in 3D Cultures

Transfected cells, e.g. porcine chondrocytes were qualitatively monitored using a Zeiss Axiovert 25. The cells were illuminated with a 50W HBO arc lamp. In the light path was a 470/40 nm excitation filter, a 495 nm beamsplitter and a 525/50 nm emission filter. Images were taken using Kodak EDAS290 directly mounted to the microscope. For quantitative measurement of expression intensity, transfected chondrocytes were measured with BMG Fluostar optima plate reader using 485/20 nm excitation filter and 535/20 emission filter.

Monitoring Luciferase Expression in 3D Cultures

Transfected cells, e.g. porcine chondrocytes were monitored using a Berthold Detection System MPL2 luminometer. Expression intensity of luciferase was measured 5 minutes after adding 100 μl PBS and 100 μl Promega's Bright-Glo™ reagent per 96-well for 10. seconds.

Monitoring Calcein-AM/Propidium Iodide Stained Cells

Cells were stained using 1 μg/ml calcein-AM and 1 μg/ml propidium iodide in phosphate buffered saline (PBS) per 2×10⁴ cells for 10 minutes. The cells were illuminated with a 50 W HBO arc lamp. In the light path was a 470/40 nm excitation filter, a 495 nm beamsplitter and a 525/50 nm emission filter to monitor life cells (green) and a 535/50 nm excitation filter, a 580 nm beamsplitter and a 590LP nm emission filter to monitor dead cells (red). Images were taken using Kodak EDAS290 directly mounted to the microscope.

Micro 3D Tissue-Like Culture Method 1-Alginate Discs

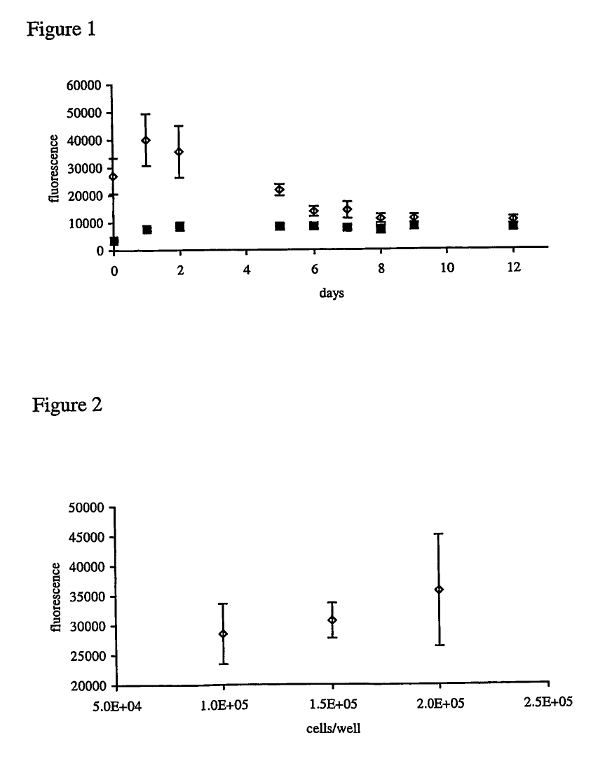

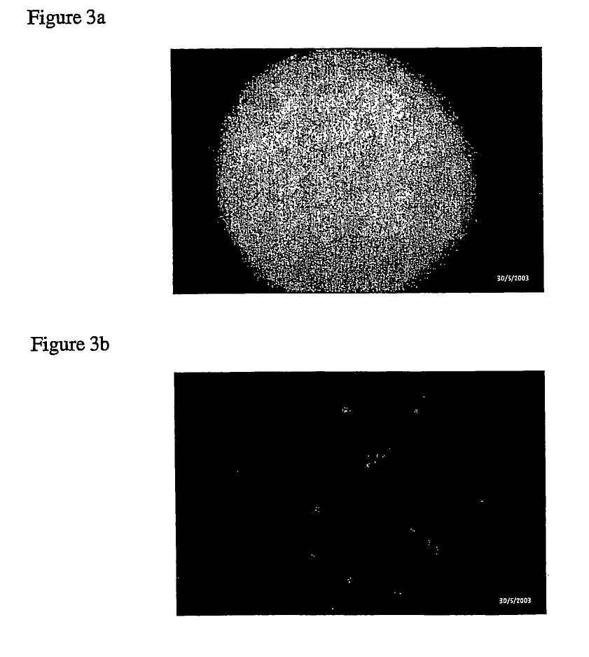

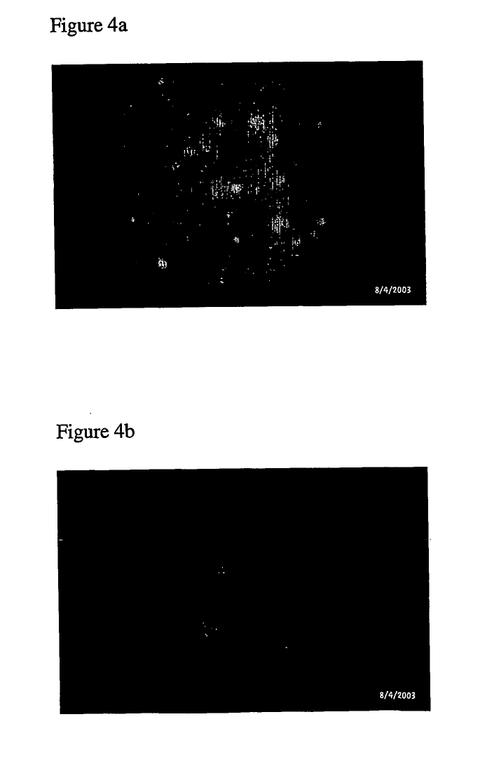

1×10⁵, 1.5×10⁵ or 2×10⁵ AV infected porcine chondrocytes from passage 2 (P2), with a transfection rate of 90%, containing the GFP gene under the control of a CMV promoter were spun and suspended in 80 μl 1.2% alginate Keltone LV dissolved in 0.9% NaCl, seeded into 96-well plates pre-coated with a 0.1 M CaCl₂-soaked isopore polycarbonate membrane filter (Millipore, Switzerland) and let be polymerized for 75 minutes at room temperature. Alginate discs were cultivated in differentiation medium as described above. GFP expression intensity was measured during 12 days using BMG Fluostar optima. Significant GFP expression can be measured during 6 days for all cell numbers used, example with 2×10⁵ cells per well can be seen in FIG. 1. GFP expression correlates with increasing cell number as can be seen e.g. on day 2 of the experiment, FIG. 2. Simultaneously, alginate discs were monitored visually with Zeiss Axiovert 25, FIG. 3. At the last day of the experiment, day 12, cells were tested for viability using calcein AM and propidium iodide staining. Over 90% viability could be observed, FIG. 4.

Micro 3D Tissue-Like Culture Method 2-Agarose Discs

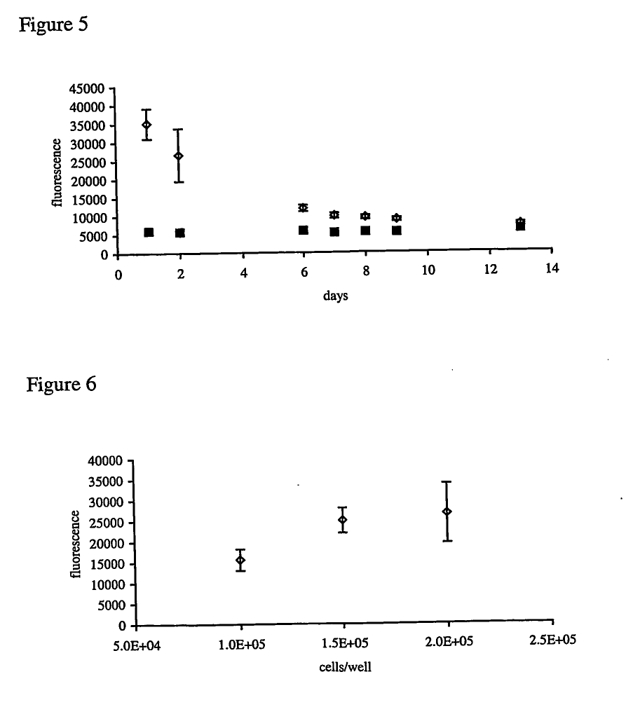

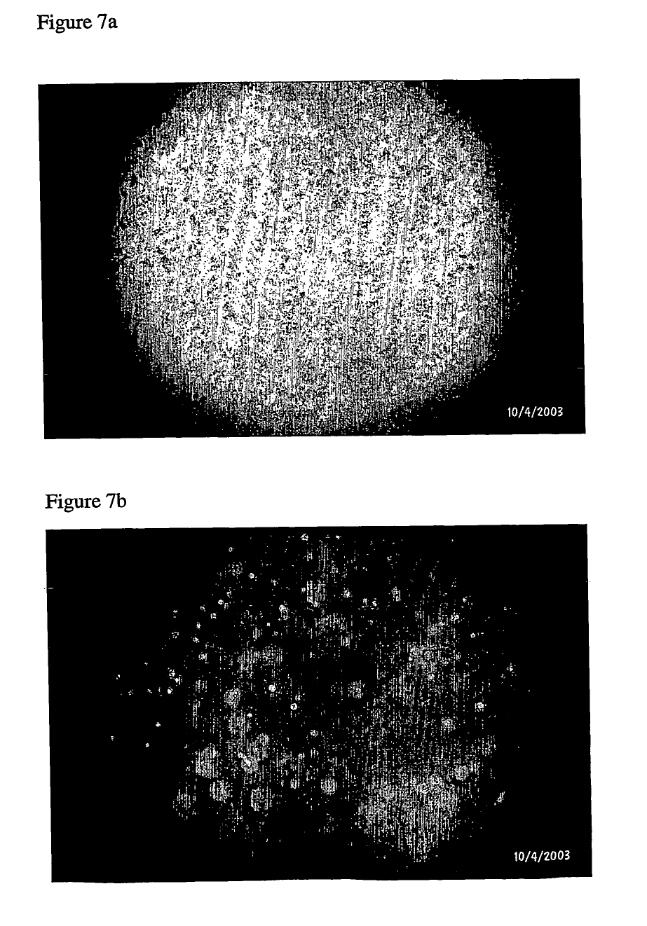

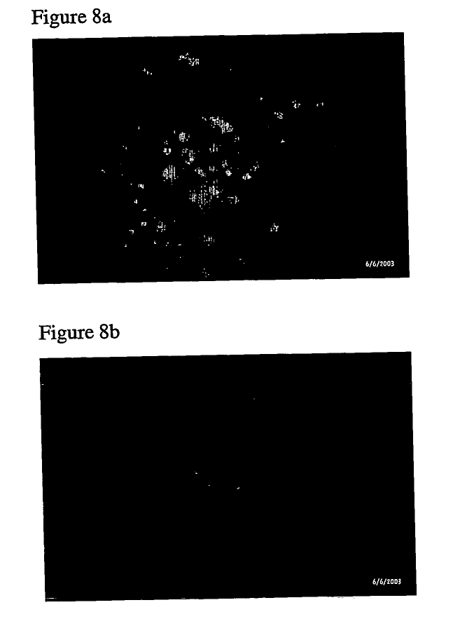

a) 1×10⁵, 1.5×10⁵ or 2×10⁵ AV infected P2 porcine chondrocytes, containing the GFP gene under the control of a CMV promoter were suspended in 20 μl DMEM/F-12, mixed with 2% agarose (low-melting, Fluka) kept at 45° C. and pipeted quickly into 96-well plates and let be gelled for 10 minutes at 4° C. Agarose discs were cultivated in differentiation medium as described above. GFP expression intensity was measured during 13 days using BMG Fluostar optima. Significant GFP expression can be measured during at least 7 days for all cell numbers used, example with 2×10⁵ cells per well can be seen in FIG. 5. GFP expression correlates with increasing cell number as can be seen e.g. on day 2 of the experiment, FIG. 6. Simultaneously, agarose discs were monitored visually with Zeiss Axiovert 25, FIG. 7. At the last day of the experiment, day 13, cells were tested for viability using calcein AM and propidium iodide staining. Over 90% viability could be observed, FIG. 8

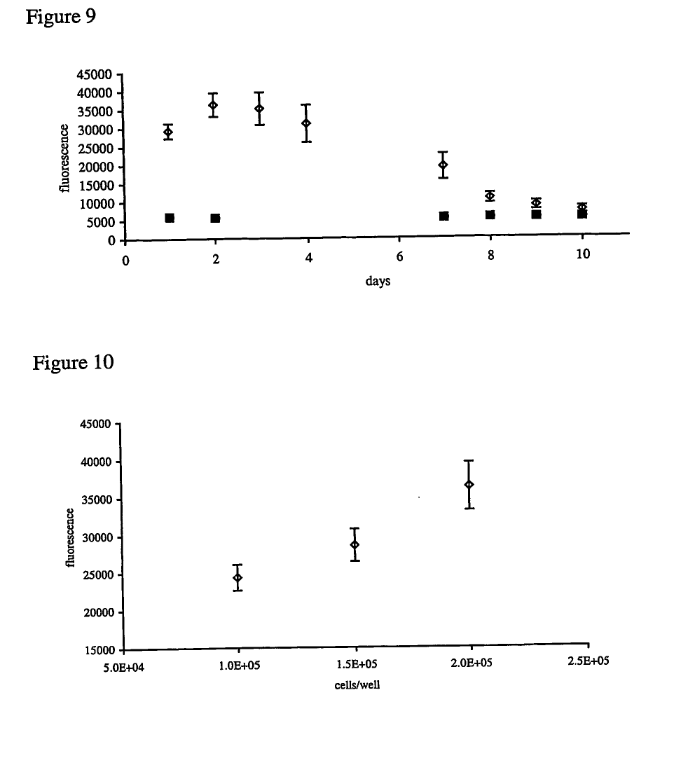

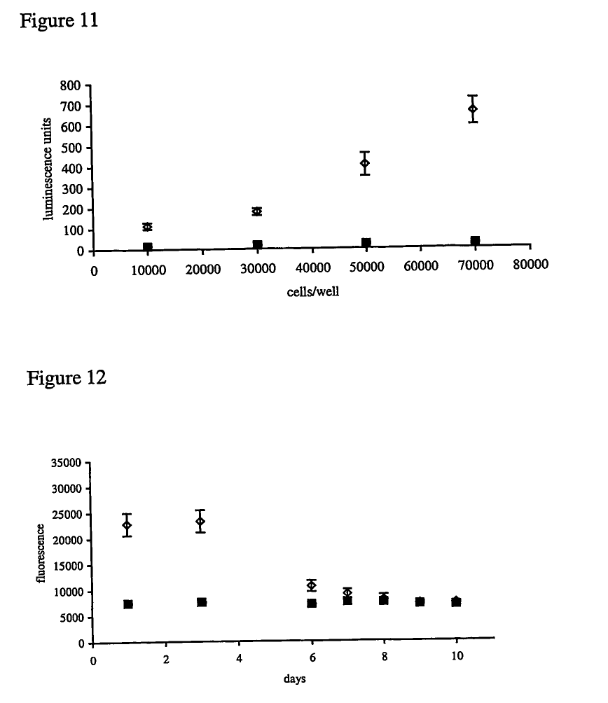

b) 1×10⁵, 1.5×10⁵ or 2×10⁵ with Amaxa Nucleofector™ technology transfected P2 human chondrocytes with a transfection rate of 40% containing the GFP gene under the control of a CMV promoter were seeded in agarose and measured for GFP expression intensity during 10 days as described above. Significant GFP expression can be measured during 8 days for all cell numbers used, example with 2×10⁵ cells per well can be seen in FIG. 9. GFP expression correlates with increasing cell number as can be seen e.g. on day 2 of the experiment, FIG. 10. At the last day of the experiment, day 10, cells were tested for viability as described above. Over 90% viability could be observed.
c) Alternatively, 1×10⁴, 3×10⁴, 5×10⁴ or 7×10⁴ with Fugene6 transfected P2 porcine chondrocytes containing the luciferase gene under the control of a COL1 promoter were seeded in agarose as described above. Luciferase expression intensity was measured as described in ‘Monitoring luciferase expression in 3D cultures’ at day 1. Transfection rate was 15%, determined as described above. FIG. 11 shows that luciferase expression correlates with increasing cell number and that only 1500 transfected cells are needed to obtain statistically relevant data. Cells were tested for viability using calcein AM and propidium iodide staining. Over 90% viability could be observed.

Micro 3D Tissue-Like Culture Method 3-polyHEMA

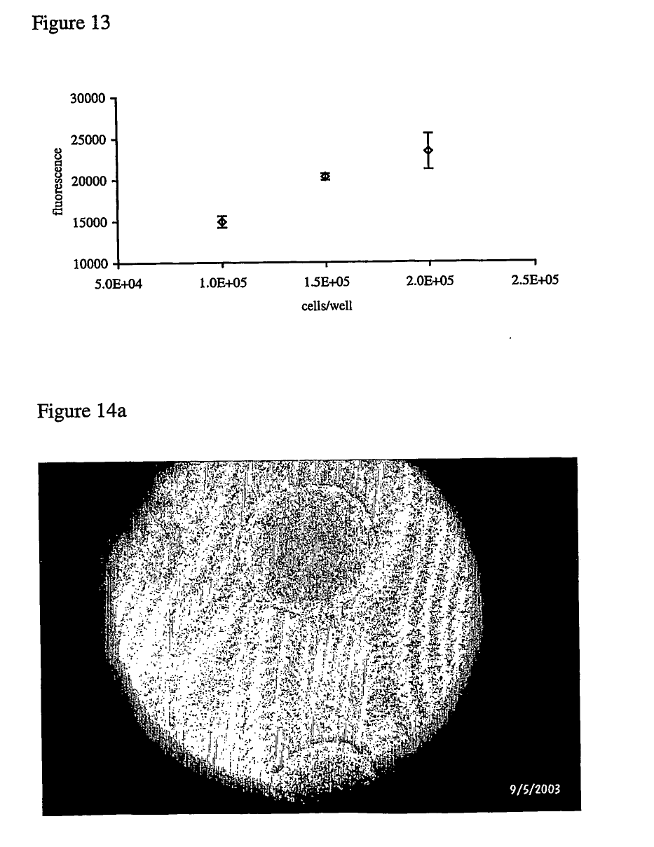

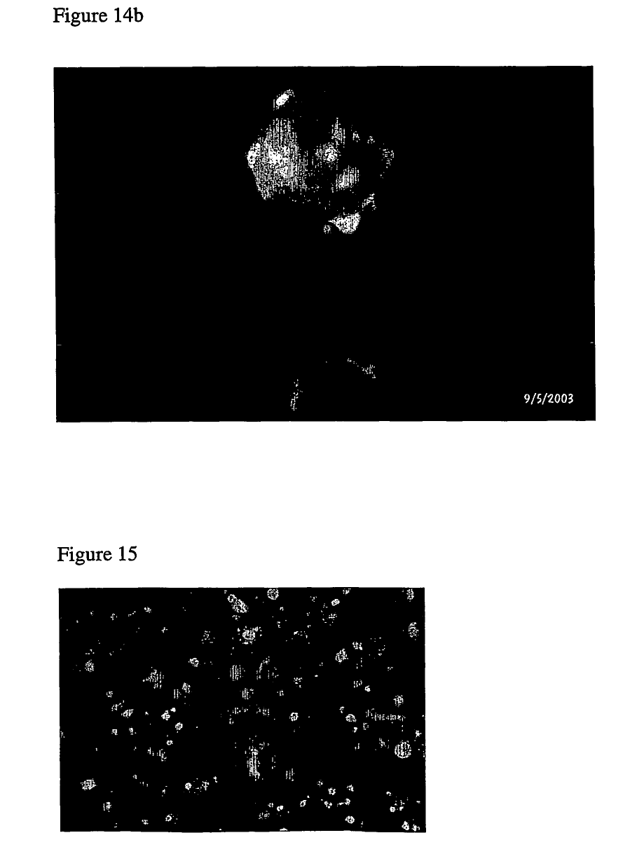

96-well plates were coated 24 hours before use with 64 μl/cm² 10% polyHEMA (Polysciences, Europe GmbH) in 95% ethanol and let be dried in sterile environment over night. 1×10⁵, 1.5×10⁵ or 2×10⁵ transfected P2 porcine chondrocytes containing the GFP gene under the control of a CMV promoter were seeded into pre-coated 96-well plates and cultivated and measured for GFP expression intensity during 10 days as described above. Significant GFP expression can be measured during 6 days for all cell numbers used, example with 2×10⁵ cells per well can be seen in FIG. 12. GFP expression correlates with increasing cell number as can be seen e.g. on day 3 of the experiment, FIG. 13. Simultaneously, cells on polyHEMA were monitored visually with Zeiss Axiovert 25, FIG. 14. At the last day of the experiment, day 10, cells were tested for viability as described above. Over 90% viability could be observed.

### EXAMPLE 2

Useful for the automated production of 3D micro cell cultures that can be used to study promoter-reporter events in biological relevant tissue-like environment using high throughput screening applications.

A suitable cell line, e.g. primary chondrocytes is expanded until the number of required cells is obtained. Cells are transfected using one of the methods described in example 1 with the promoter-reporter construct of interest, e.g. GFP under the control of COL2. Transfected cells are detached and put in a downscaled version of any of the 3D tissue-like culture systems described in example 1 using a pipeting robot. The cell solution is e.g. mixed in a ratio 1:1 with 2% agarose at a temperature of 45° C. and pipeted into a 384-well plate. For polymerization the plate is incubated for 10 minutes at 4° C. Subsequently, the plate is cultivated under standard differentiating culture conditions as described in example 1. Factors or components of the extracellular matrix, which promote the process of growing and differentiating, are added and exposed to e.g. a differentiating medium. Plates are measured automatically for GFP expression intensity using a standard fluorescence plate reader at time points of interest. Expression profile gives information about which factors or components enhance or repress extracellular matrix formation, respectively.

Any of the 3D tissue-like cell culture methods described in example 1 is suitable for downscaling and to be used within automated high throughput screening applications. Alginate solution containing transfected cells may be pipeted in 384-well plates containing isopore polycarbonate membrane membranes (Millipore, Switzerland) soaked with 0.M CaCl₂ at the bottom. To seed transfected cells on polyHEMA (Polysciences, Europe GmbH) pre-coated 384-well plates may be used.

For all systems, untransfected cells are used as control.

### EXAMPLE 3

Clinical Quality Control Tool for the Assessment of Cell-Based Therapies

Useful e.g. as quality control and diagnostic tool for cell cultures used within cell-based therapies, like e.g. autologous chondrocyte transplantation (ACT) or quality assurance of in vitro engineered constructs.

a) Human cells derived from a patient's tissue, e.g. cartilage are expanded and treated according to the cell-based therapy used. An aliquot of said cells is taken to gain knowledge about e.g. chondrogenic potential, i.e. re-differentiation of chondrocytes or the necessity of additional treatment. Cells of the taken aliquot are then transfected with one of the methods described in example 1 with a key marker promoter-reporter construct, e.g. COL2-GFP to monitor redifferentiation in chondrocytes, and are cultivated in the appropriate 3D micro tissue-like culture system. From grown constructs chondrogenic potential is assessed measuring GFP expression intensity using a standard fluorescence plate reader. The result, combined with e.g. cell viability reveals information about the chondrogenic potential and/or whether additional treatment e.g. factor adding or a complementing therapy is required.

b) To assess quality and characteristics of the cells used during in vitro production of tissue-engineered e.g. cartilage like constructs an aliquot of the proliferated cells is transfected with one of the methods in example 1 with a key marker promoter-reporter construct e.g. COL2-GFP. Subsequently the cells are cultured separately but in parallel in an appropriate 3D micro tissue-like culture system and GFP expression intensity is monitored. The chondrogenic potential is assessed accordingly and correlated with previously defined process-relevant conditions. The correlation gives information whether the to be produced constructs fulfils the required specifications.

### EXAMPLE 4

cDNA Expression Library Screening Platform Using 3D Micro Tissue-Like Cell Cultures

Useful for screening of cDNA expression libraries in 3D micro tissue-like cell culture environment.

In order to find e.g. an inducer of the collagen type II gene, CMV-driven cDNA expression libraries of interest are co-transfected with a plasmid containing the promoter of collagen type II in front of the luciferase gene into a selected cell line. The cells are cultivated in one of the 3D micro tissue-like cell culture models as described in example 1 in e.g. 96-well plates and subsequently screened for luciferase expression intensity. DNA plasmid isolation from cells that show highest luciferase expression is performed. Obtained DNA is transformed into bacteria and amplified. Plasmid is isolated and co-transfected again, the screening for highest luciferase expression is repeated. This cycle may be performed several times to be sure to isolate only plasmid containing cDNA of interest. cDNA on purified plasmid is sequenced and gene that influences promoter of interest may be identified.

### EXAMPLE 5

Useful for monitoring influence of various drugs on primary osteoarthritic cell samples, e.g. osteoarthritic chondrocytes.

Cells, e.g. osteoarthritic human chondrocytes are infected with a key marker promoter-reporter construct for osteoarthritis, e.g. aggrecanase-1 (ADAMTS4)-GFP or MMP2-GFP using a viral system to circumvent known difficulties with plasmid transfection, FIG. 15 shows highly transfected osteoarthritic chondrocytes (P2) using AV with CMV-GFP. Infected cells are seeded into a 96-well plate treated with hypothetical factors and components to assess their potential in osteoarthritis treatment, i.e. down-regulation of e.g. aggrecanase-1 or MMP2 expression. Plates are measured for GFP expression intensity using a standard fluorescence plate reader. Effectiveness of factors and components tested can be correlated to GFP signal intensity, i.e. low GFP signal equals highly efficient osteoarthritic treatment.

### EXAMPLE 6

Chondrocyte Cell Culture on Osteologic™

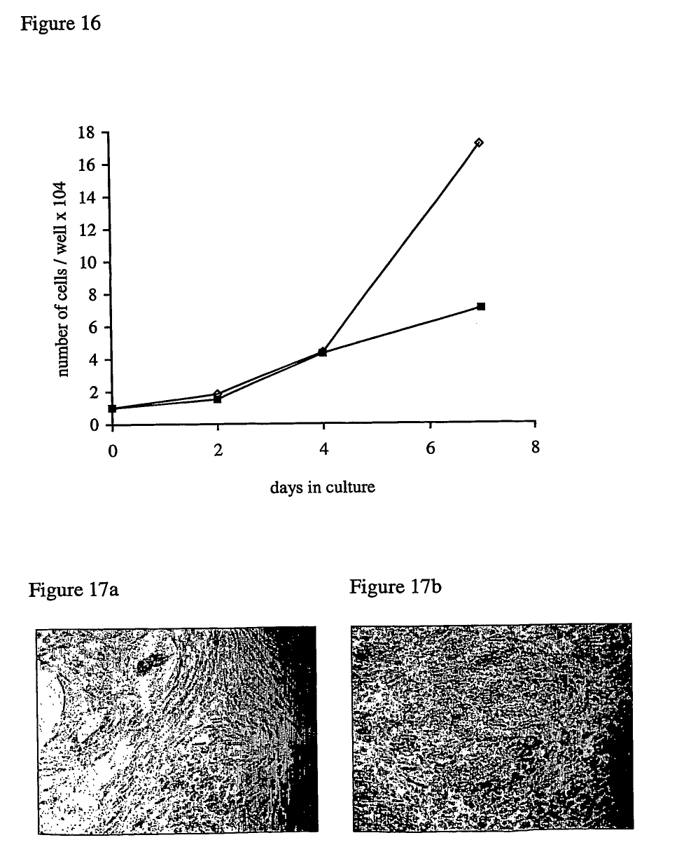

Human chondrocytes isolated from sequential enzymatic digestion of a knee biopsy were cultured in DMEM/F12 supplemented with 10% FCS and 100 IU/100 μg/ml penicillin/streptomycin. Cells were passaged once in T80 Falcon flasks harvested and seeded onto Osteologic™ discs in 24 well plates at 1×10⁴ cells per well at passage 2 (P2). Control wells were seeded directly into plastic wells without Osteologic™ discs on the same plate. Parallel plates were prepared for a time course study with cell counts taken at 0, 2, 4 & 7 days. Cells were trypsinized and counted by hemocytometer using the trypan blue method, FIG. 16. A second set of plates was plated with the same cells after culturing in flasks for an additional passage (P3). Plates were cultured for 7 days fixed with 1% gluteraldehyde in PBS and stained with a combined Periodic Acid Schiff stain (PAS) and alcian blue stain for detection of proteoglycans, FIG. 17.

### EXAMPLE 7

Human Promoter for Detection of Sox9 Expression

Useful for monitoring of sex determining region (SRY)-box containing gene 9 (SOX9) expression. SOX9 is expressed during redifferentiation in chondrocytes in 3D tissue-like culture systems.

A 750 bp fragment of the SOX9 promoter (GenBank accession number: AB022194) as described by Kanai and Koopman, 1999 is amplified with primers SOX9 sense (SEQ ID NO 1) and SOX9 antisense (SEQ ID NO 2) for genomic PCR according to standard protocols. The PCR product is digested with restriction enzymes HindIII and KpnI and ligated into pEGFP-1 (Clontech, Switzerland, GenBank accession number U55761) or into pGreenLantern (Gibco, Switzerland) digested with HindIII and KpnI. This produces a plasmid containing GFP under the control of a SOX9 promoter.

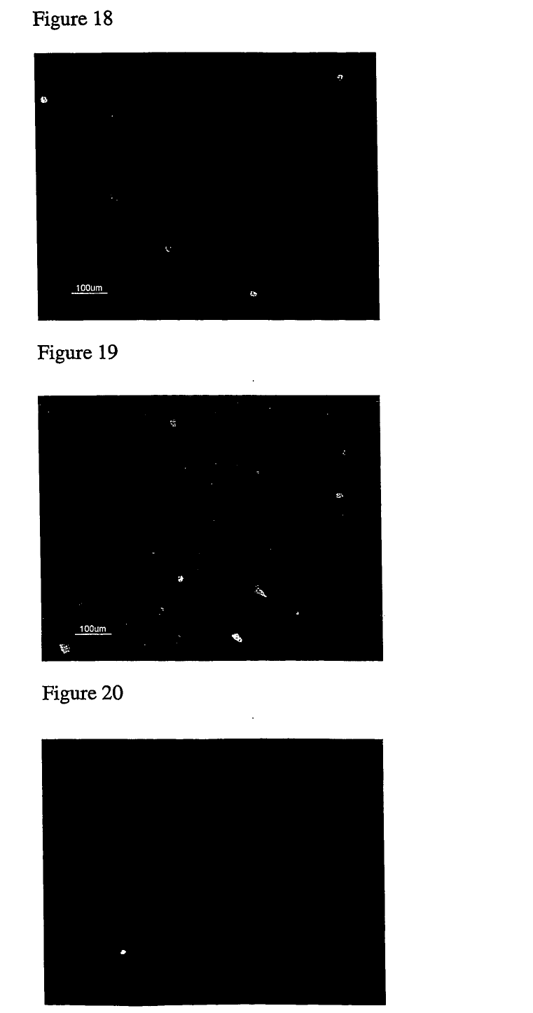

The resulting plasmid is transfected into a suitable cell line, e.g. passage 0 (P0) human chondrocytes and SOX9 expression is monitored. FIG. 18 shows that the constructed plasmid is functional.

### EXAMPLE 8

Human Promoter for Detection of Collagen Type Expression

Useful for monitoring of collagen type I (COL1) expression. COLL is expressed during dedifferentiation in chondrocytes in 2D culture systems.

A 450 bp fragment of the α2(I) collagen promoter (COL1) (GenBank accession number: AF004877) as described by Inagaki et al., 1994 is amplified from a plasmid kindly provided by F. Ramirez, New York with primers COLL sense (SEQ ID NO 3) and COL1 antisense (SEQ ID NO 4) according to standard PCR protocols. The PCR product is digested with restriction enzymes BglII and EcoRI and ligated into pEGFP-1 (Clontech, Switzerland, GenBank accession number U55761) or into pGreenLantern (Gibco, Switzerland) digested with BglII and EcoRI. This produces a plasmid containing GFP under the control of a COLL promoter.

The resulting plasmid is transfected into a suitable cell line, e.g. P0 human chondrocytes and COL1 expression is monitored. FIG. 19 shows that the constructed plasmid is functional.

### EXAMPLE 9

Human Promoter for Detection of Collagen Type II Expression

Useful for monitoring of collagen type II (COL2) expression. COL2 is expressed during redifferentiation in chondrocytes in 3D tissue-like culture systems.

A 3.785 kb fragment of the α2(I) collagen promoter (COL2) as described by Ghayor et al., 2000 is cut out from a plasmid kindly provided by L. Ala-Kokko, Oulu, Finland with restriction enzyme PdiI. The obtained fragment was ligated into pEGFP-1 (Clontech, Switzerland, GenBank accession number U55761) or into pGreenLantern (Gibco, Switzerland) digested with PdiI. This produces a plasmid containing GFP under the control of a COL2 promoter.

The resulting plasmid is transfected into a suitable cell line, e.g. P0 human chondrocytes and COL2 expression is monitored. FIG. 20 shows that the constructed plasmid is functional.

### EXAMPLE 10

Human Promoter for Detection of Cartilage Oligomeric Matrix Protein (COMP) Expression

Useful for monitoring of cartilage oligomeric matrix protein (COMP) expression. COMP is expressed during redifferentiation in chondrocytes in 3D tissue-like culture systems.

A 750 bp fragment of the COMP promoter (Genank accession number: AF069520) as described by Deere et al., 2001 is amplified with primers COMP sense (SEQ ID NO 5) and COMP antisense (SEQ ID NO 6) for genomic PCR according to standard protocols. The PCR product is digested with restriction enzymes HindIII and BamHI and ligated into pEGFP-1 (Clontech, Switzerland, GenBank accession number U55761) or into pGreenLantern (Gibco, Switzerland) digested with HindIII and BamHI. This produces a plasmid containing GFP under the control of a COMP promoter.

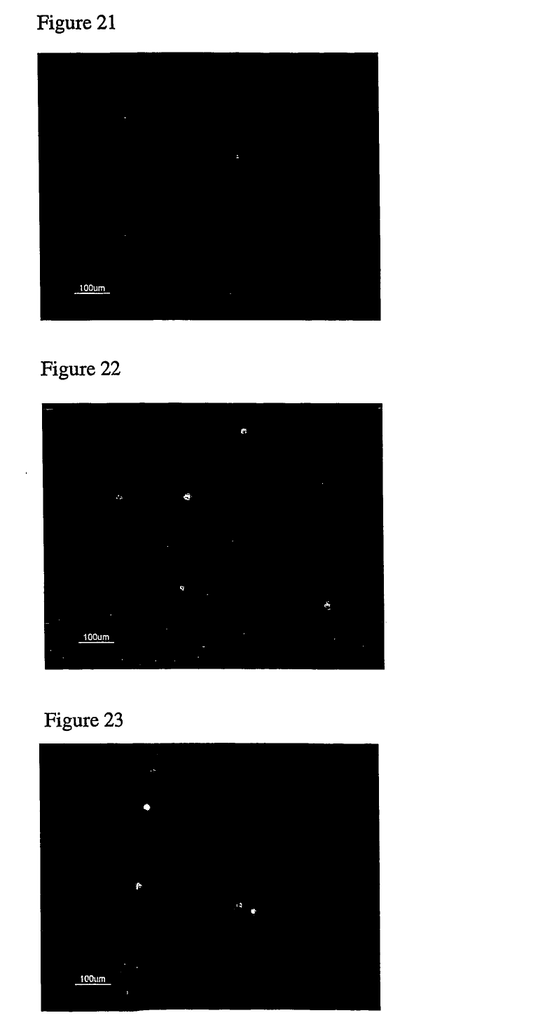

The resulting plasmid is transfected into a suitable cell line, e.g. P0 human chondrocytes and COMP expression is monitored. FIG. 21 shows that the constructed plasmid is functional.

### EXAMPLE 11

Human Promoter for Detection of Aggrecanase-1 Expression

Useful for monitoring of aggrecanase-1 (ADAMTS4) expression. Aggrecanase-1 is expressed during degradation of cartilage extracellular matrix, e.g. osteoarthritic chondrocytes.

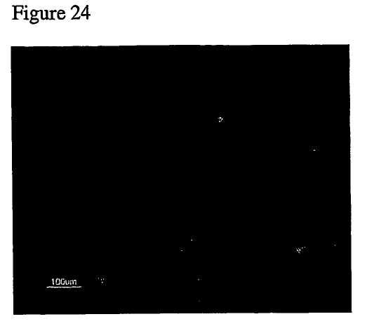

A 1.2 kb fragment of the aggrecanase-1 promoter (GenBank accession number: AB039835) as described by Mizui et al., 2000 is amplified with primers aggrecanase sense (SEQ ID NO 7) and aggrecanase antisense (SEQ ID NO 8) for genomic PCR according to standard protocols. The PCR product is cloned into PCR-Blunt II-TOPO vector (Invitrogen, Switzerland). The newly generated plasmid is digested with restriction enzymes HindIII and KpnI and the obtained aggrecanase-1 fragment is ligated into pEGFP-1 (Clontech, Switzerland, GenBank accession number U55761) or into pGreenLantern (Gibco, Switzerland) digested with HindIII and KpnI. This produces a plasmid structed plasmid containing the 1.1 kb fragment is functional. FIG. 24 shows that the constructed plasmid containing the 1.7 kb fragment is functional.

While there are shown and described presently preferred embodiments of the invention, it is to be distinctly understood that the invention is not limited thereto but may be otherwise variously embodied and practiced within the scope of the following claims.

1. A screening method for compounds having a modulating effect on cellular development and/or cell differentiation and/or cellular processes, said method comprising the following steps:
a) cultivating cells harboring a promoter-reporter construct in a 3D micro-culture under conditions mimicking the natural in vivo environment (3D tissue-like conditions) of said cells, or cultivating said cell in a 2D culture on bioinductive material,
b) contacting said cells with a test compound and comparing the read-out of the promoter-reporter construct to a control.

2. The method of claim 1, wherein said 3D tissue-like conditions comprise either 3D aggregated cells, cultivated under high cellular density only, and/or cells cultivated with natural or synthetic scaffold/biomaterial.

3. The method of claim 2, wherein said scaffolds/biomaterials are a biomaterial substrate or scaffold that promotes normal physiological activity, in particular scaffolds/biomaterials selected from the group of natural scaffolds/biomaterials consisting of alginate, agarose, hyaluronic acid, collagen, proteoglycan and mixtures thereof, or from the group of synthetic scaffolds/biomaterials consisting of Skelite™, polyHEMA, polyglycolic acid (PGA), polylactic acid (PLA) and mixtures of PGA and PLA.

4. The screening method of claim 1, wherein said cells are derived from healthy or pathological musculoskeletal tissues or precursor cells being able to differentiate and form de novo musculoskeletal tissue, preferably said cells stem from humans.

5. The method of claim 4, wherein said tissue is selected from the group consisting of chondrocytes, bone cells, rheumatoid cells, osteoarthritic chondrocytes, stem cells, mesenchymal cells, cartilage or bone tumor cells.

6. The screening method of claim 1, wherein said promoter is selected from the group consisting of human COL1, COL2, SOX9, COMP, MMP2, and aggrecanase-1 (ADAMTS4).

7. The screening method of claim 1, wherein said reporter is selected from the group of GFP, luciferase, β-galactosidase, chloramphenicol acetyltransferase gene (CAT).

8. The screening method of claim 1, wherein said cells stem from humans and said promoter-reporter construct comprises a reporter gene under control of a human promoter wherein said promoter is selected from the group consisting of human COL1, human COL2, human SOX9, human COMP, human MMP2, and human aggrecanase-1 (ADAMTS4) and said reporter gene encodes a protein with an activity that can be detected by colorimetric or fluorescent methods, in particular said reporter is selected from the group consisting of GFP, luciferase, β-galactosidase, chloramphenicol acetyltransferase gene (CAT).

9. The screening method of claim 1, wherein said cells comprise more than one promoter-reporter construct.

10. The screening method of claim 1, wherein said test compounds are selected from the group consisting of chemical libraries, natural product libraries, peptide libraries, cDNA libraries and combinatorial libraries.

11. The screening method of claim 1, wherein said method is performed in a multiplate culture format e.g. 96 or 384-mulitwells.

12. The screening method of claim 11, wherein the 3D micro cultures are produced in an automated fashion e.g. by robotic system.

13. The screening method of claim 1, wherein said cells are contacted with an activator or suppressor of said promoter and with a test compound.

14. The screening method of claim 1, wherein said method is used as a quality control tool to assess the chondrogenic potential of isolated cells prior to implantation within cell-based therapies.

15. The screening method of claim 1, wherein said method is used as a quality control tool to assess a process producing in vitro tissue-engineered cartilage constructs usable for treatment of cartilage defects.

16. The screening method of claim 1, wherein said method is used as a tool to assess the cell potency and such the suitability of cells for cell therapy and/or tissue engineered therapy.

17. A method for producing a transgenic animal comprising transforming an animal with a promoter-reporter construct wherein said reporter is selected from the group consisting of GFP, luciferase, β-galactosidase, chloramphenicol acetyltransferase gene (CAT) and said promoter is selected from the group consisting of COL1, COL2, SOX9, COMP, MMP2, and aggrecanase-1 (ADAMTS4).

18. A transgenic animal comprising a promoter-reporter construct, wherein said construct comprises a reporter selected from the group consisting of GFP, luciferase, β-galactosidase, chloramphenicol acetyltransferase gene (CAT) and a promoter selected from the group consisting of COL1, COL2, SOX9, COMP, MMP2 and aggrecanase-1 (ADAMTS4).

19. A cell line derived from the transgenic animal of claim 18.

20. A method comprising using the transgenic animal of claim 18 in a screening method for screening compounds having a modulating effect on cellular development and/or cell differentiation and/or cellular processes.

21. A DNA construct for cell transfection comprising a reporter gene under control of a human promoter wherein said promoter is selected from the group consisting of human COL1, human COL2, human SOX9, human COMP, human MMP2, and human aggrecanase-1 (ADAMTS4) and said reporter gene encodes a protein with an activity that can be detected by colorimetric or fluorescent methods.

22. The DNA construct of claim 21, wherein said reporter is selected from the group consisting of GFP, luciferase, β-galactosidase, chloramphenicol acetyltransferase gene (CAT).

23. A cell comprising a reporter construct of claim 21.

24. A cell line comprising a reporter construct of claim 21.

25. The cell or cell line of claim 23, wherein said cells are derived from healthy or pathological musculoskeletal tissues or precursor cells being able to differentiate and form de novo musculoskeletal tissue, preferably said cells stem from humans.

26. The cell or cell line of claim 25, wherein said cells are selected from the group consisting of chondrocytes, bone cells, rheumatoid cells, osteoarthritic chondrocytes, stem cells, mesenchymal cells, cartilage or bone tumor cells.

27. A method comprising performing a cellular screening assay with a cell line of claim 24.

28. A method comprising using a cell of claim 23, for the in vitro formation of tissue.

29. A method for testing whether a material has bioinductive characteristics, said method comprising the following steps:
culturing cells harboring a promoter-reporter construct on the material to be tested and comparing the read-out of the promoter-reporter construct to a control.

30. The method of claim 29, wherein said cells are human cells.

31. The method of claim 29, wherein said reporter is selected from the group consisting of GFP, luciferase, β-galactosidase, chloramphenicol acetyltransferase gene (CAT) and the promoter is selected from the group consisting of human COL1, COL2. SOX9, COMP, MMP2, and aggrecanase-1 (ADAMTS4).

32. A method for testing whether a biomaterial is degraded or resorbed in vivo or in vitro, said method comprising the following steps:
culturing cells harboring a promoter-reporter construct on the material to be tested and monitoring expression of the reporter gene in said cells.

33. The method of claim 32, wherein said cells are human cells.

34. The method of claim 32, wherein said reporter is selected from the group consisting of GFP, luciferase, β-galactosidase, chloramphenicol acetyltransferase gene (CAT) and the promoter is selected from the group consisting of human COL1, COL2, SOX9, COMP, MMP2, and aggrecanase-1 (ADAMTS4).

35. A method for the quality control of cells cultivated in vitro comprising:
transfecting cells that have been cultured in vitro with a key marker promoter-reporter construct and cultivating said transfected cells in a 3D culture and
detection of the reporter read-out which is indicative for differentiated cells.

36. The method of claim 35, wherein said cells are selected from the group consisting of chondrocytes, bone cells, rheumatoid cells, osteoarthritic chondrocytes, stem cells, mesenchymal cells, cartilage or bone tumor cells, the promoter is selected from the group consisting of GFP, luciferase, β-galactosidase, chloramphenicol acetyltransferase gene (CAT) and the promoter is selected from the group consisting of human COL1, COL2, SOX9, COMP, MMP2, and aggrecanase-1 (ADAMTS4).
